(19) United States
(12) Patent Application Publication
(10) Pub. No.: US 2007/0043118 A1
(43) Pub. Date: Feb. 22, 2007

(54) Method of treating a subject suffering from degenerative disc disease using a nitric oxide synthase inhibitor

(75) Inventor: Peter A. Zahos, Weston, FL (US)

Correspondence Address: COOPER & DUNHAM, LLP, 1185 AVENUE OF THE AMERICAS, NEW YORK, NY, 10036, US

(73) Assignee: Discogen, LLC

(21) Appl. No.: 11/505,161

(22) Filed: Aug. 15, 2006

Related U.S. Application Data
(60) Provisional application No. 60/773,666, filed on Feb. 16, 2006.
(60) Provisional application No. 60/727,552, filed on Oct. 18, 2005.
(60) Provisional application No. 60/708,430, filed on Aug. 16, 2005.

(51) Int. Cl. A61K 31/198; A61K 31/155

(52) U.S. Cl. 514/565; 514/634; 514/610

(57) ABSTRACT

The present invention provides a method for treating a vertebrate subject suffering from a degenerative disc disease by administering an inhibitor of nitric oxide synthase (NOS) to the subject in an amount effective to treat the subject.

This application claims the benefit of U.S. Provisional Applications No. 60/773,666, filed Feb. 16, 2006, Ser. No. 60/727,552, filed Oct. 18, 2005, and Ser. No. 60/708,430, filed Aug. 16, 2005, the contents of each of which is hereby incorporated by reference into this application.

Throughout this application, various references are identified by authors and full citations or by reference to the numbers of U.S. patents or Published Patent Applications. The disclosures of these references in their entireties are hereby incorporated by reference into this application to more fully describe the state of the art to which this invention pertains.

### BACKGROUND OF THE INVENTION

The human spine is formed from twenty-six consecutive vertebrae. Each of these vertebrae is separated from any adjacent vertebra by an intervertebral disc that functions to absorb shock and prevent each vertebra from directly impacting upon another vertebra. At the center of each disc is a nucleus pulposus that contains proteoglycan. Around the nucleus pulposus is an outer ring called the annulus fibrosus.

Degenerative disc disease refers to any of the common degenerative conditions of the lower spine involving degeneration of the disc. Disc degeneration is often associated with the symptom of pain and may lead to inflammation and neuropathic pain, for example, spinal stenosis, spondylolisthesis, and retrolisthesis.

Disc degeneration associated with the aging process is generally associated with the loss of proteoglycan from the nucleus pulposus of the spinal discs and a reduction of the disc's ability to absorb shock between vertebrae.

Although some affected patients may not exhibit symptoms, many affected patients suffer from chronic back and/or leg pain. Pain associated with disc degeneration may become debilitating and may greatly reduce a patient's quality of life.

While nonoperative treatments for disc degeneration exist, many patients, for example those patients with severe symptoms, may not respond to nonoperative treatment. Conventional operative treatment generally involves spondylosyndesis (spinal fusion), which is highly invasive and is associated with certain risks to the patient.

### SUMMARY OF THE INVENTION

This invention provides a method for treating a vertebrate subject suffering from a degenerative disc disease which involves administering to the subject an inhibitor of nitric oxide synthase in an amount effective to treat the subject. Examples of such an inhibitor include GED, L-NAME, and L-NMMA.

This invention also provides a method for alleviating symptoms of a degenerative disc disease in a subject which comprises administering to the subject an effective symptom alleviating amount of an inhibitor of nitric oxide synthase.

### DETAILED DESCRIPTION OF THE INVENTION

The present invention provides a method for treating a vertebrate subject suffering from a degenerative disc disease which comprises administering to the subject an inhibitor of nitric oxide synthase in an amount effective to treat the subject.

In one embodiment of the invention, the amount effective to treat the subject is an amount effective to slow or preferably to inhibit progression of the disc degeneration disease

In another embodiment of the invention, the amount effective to treat the subject is an amount effective to provide symptomatic relief to the subject, for example, to reduce the pain associated with the degenerative disc disease.

In connection with the invention, the inhibitor is preferably a compound having the chemical structure:

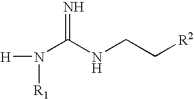

wherein R₁ represents a C₁-C₆ alkyl, C₂-C₆ alkenyl, C₂-C₆ alkynyl, OH, H, —NO₂, or halide group; and R₂ represents a substituted or unsubstituted C₁-C₃₀, straight chain or branched alkyl group, wherein the alkyl, if substituted, is substituted by a hydroxy, carboxy, amino, acetoxy, or nitroxy group or a pharmaceutically acceptable salt or ester thereof.

In one presently preferred embodiment of the invention, the inhibitor is guanidinoethyldisulfide (GED) which has the structure:

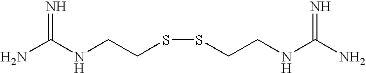

or a pharmaceutically acceptable salt or ester thereof.

In another presently preferred embodiment of the invention, the inhibitor is nitro-L-arginine methyl ester (L-NAME) which has the structure:

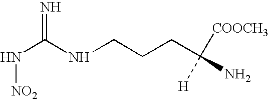

or a pharmaceutically acceptable salt or ester thereof.

In yet another presently preferred embodiment of the invention, the inhibitor is N-monomethyl-L-arginine (L-NMMA) which has the structure:

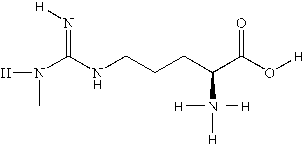

or a pharmaceutically acceptable salt or ester thereof.

Typically, the inhibitor is administered as a component of a composition which comprises a pharmaceutically acceptable carrier.

In one embodiment of the invention, the pharmaceutically acceptable carrier comprises a biopolymer, such as hyaluronic acid or a pharmaceutically acceptable salt or ester thereof, e.g. sodium hyaluronate.

In such a composition, the inhibitor is present within the composition at a concentration between 0.1% and 5.0% by weight, preferably, between 0.1% and 1.0% by weight.

In the practice of the invention, the inhibitor is typically administered in conjunction with a surgical procedure which results in the intervertebral discs of the subject being accessible to the inhibitor, e.g. laminectomy or microdiscectomy. Alternatively, the inhibitor is administered by means of a needle or a cannula.

In connection with the invention, the amount of the inhibitor administered to the subject is generally between 0.1 mg/kg and 30 mg/kg body weight of the subject. For certain inhibitors the amount is preferably between 0.3 mg/kg and 10 mg/kg body weight of the subject and the concentration of the inhibitor is typically between 0.1 to 1000 micromolar, preferably between 1.0 to 100 micromolar.

For other inhibitors, the amount of the inhibitor administered to the subject is generally between 1.0 mg/kg and 30 mg/kg body weight of the subject, preferably between 1.0 mg/kg and 10 mg/kg body weight of the subject and the concentration of the inhibitor is typically between 1.0 to 1000 micromolar, preferably between 5.0 to 100 micromolar.

In practice the inhibitor may be administered at any given site once daily or more typically periodically, e.g. weekly, biweekly or monthly over a period of 30-180 days.

As used herein, a “vertebrate subject” means any subject having a segmented spinal column. Examples include mammals, for example a pig, rat, or dog, and primates, for example a monkey, chimpanzee, orangutan, or a human.

As used herein, a “degenerative disc disease” means any condition which results in degeneration of an intervertebral disc, including but not limited to, conditions caused by a disease, physical impact, mechanical wear, a pathogen, or an autoimmune response.

As used herein, “nitric oxide synthase” means a naturally occurring enzyme which catalyzes in vivo synthesis of nitric oxide.

As used herein, “biopolymer” means any polymeric chemical manufactured by a living organism, e.g. proteins and polysaccharides as well as such substances when prepared by chemical syntheses. Many biopolymers spontaneously “fold” into characteristic shapes (also referred to as secondary structure and tertiary structure), which determine their biological function and depend in a complicated way on the primary structure of the biopolymer.

As used herein, a “salt” means any salt of the compounds useful as nitric oxide inhibitor which has been modified by making acid or base salts of the compounds, prefurably pharmaceutically acceptable salts. Examples of pharmaceutically acceptable salts include, but are not limited to, mineral or organic acid salts of basic residues such as amines; alkali or organic salts of acidic residues such as carboxcylic acids. The salts can be made using an organic or inorganic acid. Such acid salts include chlorides, bromides, sulfates, nitrates, phosphates, sulfonates, formates, tartrates, maleates, malates, citrates, benzoates, salicylates, ascorbates, and the like. Carboxylate salts are the alkaline earth metal salts, sodium, potassium or lithium.

As used herein, “alkyl” means and includes both branched and straight-chain saturated aliphatic hydrocarbon groups having the specified number of carbon atoms. Thus, C₁-Cₙ as in “C₁-Cₙ alkyl” includes groups having 1, 2, . . . , or n carbons in a linear or branched arrangement, and specifically includes methyl, ethyl, propyl, butyl, pentyl, hexyl, and so on. For example, C₁-C₆, as in “C₁-C₆ alkyl”, is defined to include individual moieties having 1, 2, 3, 4, 5, or 6 carbons in a linear or branched arrangement. “Alkoxy” represents an alkyl moiety of indicated number of carbon atoms which is attached to a core structure through an oxygen bridge or bond.

The term “cycloalkyl” means cyclic rings of alkanes of three to eight carbon atoms, e.g., cyclopropyl, cyclobutyl, cyclopentyl, cyclohexyl, cycloheptyl or cyclooctyl.

If no number of carbon atoms is specified, the term “alkenyl” refers to a non-aromatic hydrocarbon radical, straight or branched, containing at least 1 carbon to carbon double bond, and up to the maximum possible number of non-aromatic carbon-carbon double bonds may be present. If the number of carbon atoms is specified, e.g. “C₂-Cₙ” alkenyl, each member of the numeric range is disclosed individually as discussed above. Thus, for example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “cycloalkenyl” means cyclic rings of 3 to 10 carbon atoms and at least 1 carbon to carbon double bond (i.e., cyclopropenyl, cyclobutenyl, cyclopenentyl, cyclohexenyl, cycloheptenyl or cycloocentyl).

The term “alkynyl” refers to a hydrocarbon radical straight or branched, containing at least 1 carbon to carbon triple bond, and up to the maximum possible number of non-aromatic carbon-carbon triple bonds may be present. Thus, “C₂-C₆ alkynyl” means an alkynyl radical having 2, 3, 4, 5, or 6 carbon atoms, and for example 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated.

In differing embodiments of alkyl as used herein the alkyl is a C1-C10 alkyl. In differing embodiments of alkenyl as used herein the alkenyl is a C2-C10 alkenyl. In differing embodiments of alkynyl as used herein the alkenyl is a C2-C10 alkynyl.

As used herein, “aryl” is intended to mean any stable monocyclic, bicyclic or tricyclic carbon ring of up to 10 atoms, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic or bicyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

“Halo” or “halogen” as used herein is intended to include chloro, fluoro, bromo and iodo.

The term “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition. The alkyl, alkenyl, alkynyl, cycloalkyl, aryl, heteroaryl and heterocyclyl substituents may be substituted or unsubstituted, unless specifically defined otherwise. For example, a (C1-C7) alkyl may be substituted with one or more substituents selected from OH, oxo, halogen, alkoxy, dialkylamino, or heterocyclyl, such as morpholinyl, piperidinyl, and so on. In the compounds of the present invention, alkyl, alkenyl, alkynyl, cycloalkyl, cycloalkenyl, heterocyclyl and heteroaryl groups can be further substituted by replacing one or more hydrogen atoms by alternative non-hydrogen groups. These include, but are not limited to, halo, hydroxy, mercapto, amino, carboxy, cyano and carbamoyl.

The term “substituted” includes multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In embodiments of this invention, unsubstituted substituted aromatic rings include six-membered rings. In an embodiment the ring is substituted by a C1-C10 alkyl, alkenyl or alkynyl, each of which may be linear or branched, and each of which may be substituted themselves with one or more amino groups. In an embodiment the substituted pyrroles groups of this invention are substituted by a C1-C10 alkyl, alkenyl or alkynyl, each of which may be linear or branched, and each of which may be substituted themselves with one or more amino groups. In one embodiment the pyrrole group is substituted with an ethylamino group.

In an embodiment the alkyl, alkenyl or alkynyl, alkylene, alkenylene or alkynlene groups of this invention have 1, 2, 3, 4, 5, 6, 7, 8, 9 or 10 carbon atoms. In choosing compounds of the present invention, one of ordinary skill in the art will recognize that the various substituents, i.e. R¹, R², and R³, are to be chosen in conformity with well-known principles of chemical structure connectivity.

This invention will be better understood by reference to the Examples which follow, but those skilled in the art will readily appreciate that these specific examples are only illustrative of the invention as defined by the claims which follow thereafter.

It will be noted that the structures of certain compounds useful in this invention include asymmetric carbon atoms and thus occur as racemates and racemic mixtures, single enantiomers, diastereomeric mixtures and individual diastereomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention. Such isomers can often be obtained in substantially pure form by classical separation techniques and by stereochemically controlled synthesis procedures.

As set out above, certain compounds useful in this invention contain a basic functional group, such as an amino or alkylamino group, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The term “pharmaceutically acceptable salts” as used herein also includes a quaternary ammonium salt.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for subcutaneous administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect.

Methods of preparing these formulations or compositions include the step of bringing into association a compound of the present invention with the carrier and, optionally, one or more accessory ingredients are well known in the art. In general, the formulations are prepared by uniformly and intimately bringing into association a compound of the present invention with liquid carriers, or finely divided solid carriers, or both, and then, if necessary, shaping the product.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein mean modes of administration other than enteral and topical administration, usually by injection, and include, without limitation, intradiscal, intravenous, intramuscular, intraarterial, intrathecal, intracapsular, intraorbital, intracardiac, intradermal,intraperitoneal, transtracheal, subcutaneous, subcuticular, intraarticular, subcapsular, subarachnoid, intraspinal and intrastemal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage levels of the active ingredients in the pharmaceutical compositions used in the method of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound in the method of the invention will be that amount of the compound which is the lowest dose effective to produce a therapeutic effect. Such an effective dose will generally depend upon the factors described above.

If desired, the effective total dose of the active compound may be administered as two, three, four, five, six or more sub-doses administered separately at appropriate intervals, optionally, in unit dosage forms.

Administration may be to multiple spinal segments. Where multiple spinal segments are involved, the treatment may be administered to all involved spinal segments in a single treatment session or at various times during a single day or a longer period of time. Administration to spinal segments may occur on multiple occasions or may be periodically performed, for example, once a month, semiannually, or once a year.

Lower back pain, with or without radiculopathy, continues to be a significant clinical symptom causing major disability in patients. Degeneration of the intervertebral discs has been implicated as one of the factors associated with lower back pain and is a probable prerequisite to disc herniations, which are associated most commonly with acute radicular syndromes. (See, for example, “Kang et al. I”, Herniated Lumbar Intervertebral Discs Spontaneously Produce Matrix Metalloproteinases, Nitric Oxide Interleukin-6 and Prostaglandin E₂, James D. Kang, MD, et al., Spine, Volume 21, Number 3, pp 271-277, 1996.

In the present invention disc degeneration is treated in mammalian subjects. Specifically, regenerative therapies comprising biological agents act to inhibit nitric oxide synthase (NOS) and treat disc degeneration. The regenerative therapy is delivered to the intervertebral disc and inhibits proteoglycan loss by interfering with the biochemical cascade that leads to disc degeneration.

Accordingly, the regenerative therapy comprises one or more NOS inhibitors in an amount effective to treat the subject. By treating the subject, the progress of disc degeneration is slowed or stopped.

NOS inhibitors reduce proteoglycan loss by interfering with the biochemical cascade that leads to disc degeneration.

Intervertebral disc is an avascular tissue populated by poorly characterized cells in an extensive extracellular matrix. The matrix of the central nucleus pulposus is rich in proteoglycans, whereas the anulus fibrosus is predominantly collagenous. With aging, the content of proteoglycans significantly decreases thereby contributing to disc degeneration. (See Kang et al. I).

The excessive production of nitric oxide (NO) has been shown to exert cytotoxic effects on various human cells. NOS, which is responsible for producing large amounts of NO may be present in a variety of cells, particularly following injury and certain other inflammatory stimuli. See, for example, Pseudoguaianolides Isolated from Inula britnnica var. chinenis as inhibitory Constituents against Inducible Nitric Oxide Synthase, Hyun-Tai Lee et al., Arcg Pharm Res Vol 25, No 2, pp 151-153, 2002 which. Accordingly, NOS inhibitors are believed to be effective therapeutic agents for pathological conditions related to NO.

There is also evidence to support that NOS may play a role in the pathophysiology of autoimmune and/or inflammatory conditions such as arthritis, rheumatoid arthritis and systemic lupus erythematosus and insulin-dependent diabetes mellitus. There is also evidence to support that a number of inflammatory and non-inflammatory diseases are associated with NO overproduction. See, for example, U.S. Pat. No. 5,922,756 to Chan.

Matrix metalloproteinases (MMPs), prostaglandin E₂ (PGE₂), and a variety of cytokines have been shown to play a role in the degeneration of articular cartilage. Nitric oxide (NO) is a novel mediator that is implicated in cartilage abnormalities. (See Kang et al. I).

Nitric oxide (NO) is synthesized from the guanidino group of L-arginine by a family of enzymes termed nitric oxide synthase (NOS). The brain isoform (BNOS) is continuously present in the neural tissue and NO is released as a neurotransmitter by activation of various (e.g. NMDA-type) receptors. NO in the central nervous system plays an important role in the genesis of memory.

The continuous release of NO from the constitutive endothelial isoform of NOS (ecNOS) keeps the vasculature in a continuous state of active vasodilatation and reduces the adhesion of platelets and polymorphonuclear granulocytes (PMNs) to the endothelial surface. The biological activity of NO from the ecNOS was originally described as endothelium-derived relaxing factor (EDRF). The release of EDRF in vivo and in vitro is stimulated by shear stress and various hormones and autocoids such as acetylcholine, bradykinin, substance P. vasopressin, noradrenaline, histamine or platelet-activating factor.

The inducible isoform of NOS (iNOS) is expressed in response to immunological stimuli in multiple cell types including macrophages, vascular smooth muscle cells and epithelial cells, and produces large amounts of NO (nanomoles of NO rather than picomoles of NO derived by the ecNOS or bNOS). NO in high local concentrations can act as a cytostatic and cytotoxic molecule acting against fungal, bacterial, helminthic and protozoal antigens as well as tumor cells. A number of pro-inflammatory cytokines and endotoxin (bacterial lipopolysaccharide, LPS) also induce the expression of iNOS in a number of other cells, including fibroblasts, glial cells, cardiac myocytes as well as vascular and non-vascular smooth muscle cells.

There is now substantial evidence that iNOS plays an important role in the pathogenesis of a variety of diseases. Circulatory shock of various etiologies is associated with profound changes in the body's NO homeostasis. In animal models of endotoxic shock, endotoxin produces an acute release of NO from the constitutive isoform of nitric oxide synthase in the early phase, which is followed by induction of iNOS. In addition, it is now thought that excess NO production may be involved in a number of conditions, including conditions that involve systemic hypotension such as septic (toxic) shock and therapy with certain cytokines. Therefore, it is desirable to inhibit nitric oxide synthase. Furthermore, because of the potentially serious consequences of over-inhibition of the constitutive NOS enzyme, it is preferred to selectively inhibit the inducible isoform. Over-inhibition of the constitutive isoform may lead to hypertension, thrombosis, central nervous system toxicity and tissue damage.

NO synthase enzyme may be involved in the pathophysiology of autoimmune and/or inflammatory conditions such as arthritis, rheumatoid arthritis and systemic lupus erythematosus (SLE) and in insulin-dependent diabetes mellitus, and therefore, mercapto derivatives may prove helpful in treating these conditions.

Furthermore, there are a number of additional inflammatory and noninflammatory diseases that are associated with NO overproduction. Examples of such physiological disorders include: inflammatory bowel diseases such as ileitis, ulcerative colitis and Crohn's disease; inflammatory lung disorders such as asthma and chronic obstructive airway disease; inflammatory disorders of the eye including corneal dystrophy, trachoma, onchocerciasis, uveitis, sympathetic ophthalmitis and endophthalmitis; chronic inflammatory disorders of the gum including periodontitis; chronic inflammatory disorders of the joints including arthritis and osteoarthritis, tuberculosis, leprosy, glomerulonephritis sarcoid, and nephrosis; disorders of the skin including sclerodermatitis, psoriasis and eczema; inflammatory diseases of the central nervous system, including chronic demyelinating diseases such as multiple sclerosis, dementia including AIDS-related neurodegeneration and Alzheimer's disease, encephalomyelitis and viral or autoimmune encephalitis; autoimmune diseases including immune-complex vasculitis, systemic lupus and erythematodes; and disease of the heart including ischemic heart disease and cardiomyopathy. Additional disease that may benefit from the use of mercapto derivatives include adrenal insufficiency; hypercholesterolemia; atherosclerosis; bone disease associated with increased bone resorption, e.g., osteoporosis, pre-eclampsia, eclampsia, uremic complications; chronic liver failure, noninflammatory diseases of the central nervous system (CNS) including stroke and cerebral ischemia; and various forms of cancer.

NO also plays a role in wound healing. Wound healing involves the recruitment of inflammatory cells, followed by fibroblasts, to the site of the wound, where collagen and other connective tissue elements are deposited. The collagen fibers then gradually realign to resemble the original connective tissue (e.g. tendon, ligament, skin.) The ability to regulate this process locally and specifically would be of considerable therapeutic importance e.g. after surgery or trauma. Furthermore, in certain pathological situations, such as arthrofibrosis, Dupuytren's contracture, peritoneal adhesions, frozen shoulder, scleroderma, and keloid formation, over-expression, and sometimes normal expression, of the repair mechanisms has negative consequences, and it would be desirable to selectively suppress this response.

Conversely, there are many situations in which the healing response in wound healing is delayed or inhibited e.g. in patients with systemic diseases such as liver failure, renal impairment, diabetes, peripheral vascular disease, or in patients taking drugs that inhibit healing e.g. corticosteroids or immunosuppressive agents. In these cases, additional exogenous NO may enhance the healing response.

The clinical presentation of acute lumbar radiculopathy is most often attributed to a compressed lumbar nerve root by a herniated intervertebral disc. Some patients with large herniations have no radicular symptoms, and, in contrast, some patients with no evidence of disc herniations have severe radiculopathy. Direct mechanical compression of a nerve root and biochemical mediators of inflammation each play a role in disc degeneration and radicular pain. (See Kang et al. I).

Biochemical mediators of inflammation and tissue degeneration play a role in intervertebral disc herniations and the pathophysiology of radiculopathy. (See Kang et al. I).

Culture media from herniated lumbar discs show increased levels of matrix metalloproteinase activity in comparison with control discs. Similarly, levels of nitric oxide, prostaglandin E₂, and interleukin-6 are significantly higher in herniated discs in comparison with control discs. Accordingly, herniated lumbar discs make spontaneously increased amounts of matrix metalloproteinases, nitric oxide, prostaglandin E₂, and interleukin-6. These products are intimately involved in the biochemistry of disc degeneration and the pathophysiology of radiculopathy. (See Kang et al. I).

Traditionally, disc degeneration has been considered to be largely a biochemical phenomenon. However, biochemical mechanisms are believed to play a much larger role than is generally appreciated. Articular cartilage provides a good example of how a cartilaginous tissue is vulnerable to the influences of biologic stimuli. For instance, in articular cartilage, interleukin-1 (IL-1) inhibits proteoglycan synthesis through a mechanism that, at least partly, involves the induction of nitric oxide (NO) synthesis. (See, for example, “Kang et al. II”, Toward a Biochemical Understanding of Human Intervertebral Disc Degeneration and Herniation, James D. Kang, MD, et al., SPINE Volume 22, Number 10, pp 1065-1073, 1997.

Based on their roles in articular cartilage pathophysiology, NO and other biological agents are believed to be involved in the net loss of proteoglycans associated with disc degeneration.

Certain NOS inhibitors, for example L-NMMA, have been demonstrated to reduce the level of NO present, in vitro, in disc tissue obtained from human subjects. (See Kang et al. I) Embodiments of the present invention seek to utilize various NOS inhibitors, for example GED, L-NAME, and L-NMMA to reduce NO production in vivo, within the discs of a patient suffering from disc degeneration.

Studies have shown that chronic lower back pain may be treated by direct intervertebral injection of biological agents. See, for example, Biochemical Injection Treatment for Discogenic Low Back Pain: a Pilot Study, Robert G. Klein, MD, Björn C. J. Eek, MD, Connor W. O'Neill, MD, Caren Elin, DC, Vert Mooney, MD, Richard R. Derby, MD, The Spine Journal 3, pp 220-226, 2003.

Based on the associations between NOS and disc degeneration, the present invention utilizes NOS inhibitors to interfere with the process of disc degeneration. The use of NOS inhibitors inhibits proteoglycan loss and/or reduces inflammation and neuropathic pain associated with disc degeneration. This is because NOS plays a regulatory role in the interaction between the biochemical agents produced by degenerated discs. Additionally, NO production is a key modulator in proteoglycan loss and development of neuropathic pain.

Because disc degeneration is associated with the inflammatory cascade, it is contemplated that the inhibition of NOS would interfere with the progression of disc degeneration.

Pharmacologically acceptable compositions for inhibiting NOS in mammals are known in the art. For example, U.S. Pat. No. 5,952,385 and U.S. Pat. No. 5,985,917 to Southan et al., (the “Southan patents”) describe compositions including a mercapto or seleno derivative effective to inhibit nitric oxide synthase in the mammal.

Alternatively or additionally, other known NOS inhibitors may be used. For example, guanidinoethyldisulfide (GED) or a pharmaceutically acceptable salt or ester thereof may be used as a NOS inhibitor.

Below is the chemical structure for GED:

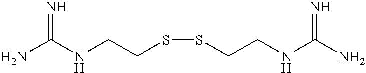

Some mercaptoalkylguanidines, for example, mercaptoethylruanidine (MEG) and its dimeric form, guanidinoethyldisulphide (GED) have been demonstrated to be a novel class of NOS inhibitors, see, for example, Pharmacological characterization of guanidinoethyldisulphide (GED), a novel inhibitor if nitric oxide synthase with selectivity towards the inducible isoform, Csaba Szabo et al., Br J Pharmacol. 1996 August;118(7):1659-68.

Therapeutic agents formulated with GED according to embodiments of the present invention may be preferably dosed within the range of 0.1 mg/kg to 30 mg/kg by weight; 0.1 to 1000 micromolar by concentration and more preferably within the range of 0.3 to 10 mg/kg by weight; 1.0 to 100 micromolar by concentration.

The pharmaceutical formulations of the present invention need not in themselves contain the entire amount of the agent needed to be effective; as such effective amounts can be reached by administration of a single application or dose, or a plurality of applications or doses of such pharmaceutical formulations.

Alternatively or additionally, other known NOS inhibitors may be used. For example, nitro-L-arginine methyl ester (L-NAME) or a pharmaceutically acceptable salt or ester thereof may be used as a NOS inhibitor.

Below is the chemical structure for L-NAME:

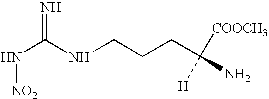

Therapeutic agents formulated with L-NAME according to embodiments of the present invention may be preferably dosed within the range of 0.1 mg/kg to 30 mg/kg by weight; 1.0 to 1000 micromolar by concentration and more preferably within the range of 1.0 mg/kg to 10 mg/kg by weight; 5.0 to 100 micromolar by concentration.

The pharmaceutical formulations of the present invention need not in themselves contain the entire amount of the agent needed to be effective; as such effective amounts can be reached by administration of a single application or dose, or a plurality of applications or doses of such pharmaceutical formulations.

Alternatively or additionally, other known NOS inhibitors may be used. For example, N-monomethyl-L-arginine (also known as N_{ω}-Methyl-L-arginine, N^{G}-Monomethyl-L-arginene, and L-NMA) (herein also referred to as L-NMMA) or a pharmaceutically acceptable salt or ester thereof may be used as a NOS inhibitor.

Below is the chemical structure for L-NMMA:

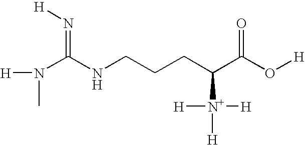

Therapeutic agents formulated with L-NAME according to embodiments of the present invention may be preferably dosed within the range of 0.1 mg/kg to 30 mg/kg by weight; 1.0 to 1000 micromolar by concentration and more preferably within the range of 1.0 to 10 mg/kg by weight; 5.0 to 100 micromolar by concentration.

The pharmaceutical formulations of the present invention need not in themselves contain the entire amount of the agent needed to be effective; as such effective amounts can be reached by administration of a single application or dose, or a plurality of applications or doses of such pharmaceutical formulations.

Each of the above-identified NOS inhibitors share a common generic structure and it is believed that all chemicals sharing the same common generic structure can be used for the treatment of disc degeneration according to embodiments of the present invention.

Below is the common generic chemical structure:

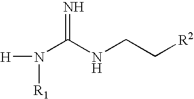

In which R_{1 is C}₁-C₆ alkyl, C₂-C₆ alkenyl, C₂-C₆ alkynyl, OH, H, —NO₂, or halide; and R₂ is a substituted or unsubstituted, straight chain C₁-C₃₀ alkyl or branched C₁-C₃₀ alkyl wherein the alkyl, if substituted, is substituted by hydroxy, carboxy, amino, acetoxy, or nitroxy.

Therapeutic agents formulated with the generic structure above according to embodiments of the present invention may be preferably dosed within the range of 0.1 to 30 mg/kg by weight and more preferably within the range of 0.3 to 10 mg/kg.

In addition to the above-mentioned inhibitors of NOS, including GED, L-NAME, and L-NMMA, other known NOS inhibitors may be used according to the present invention. For example, other NOS inhibitors of the inducible isoform may be used, including isothiourea derivatives and aminoguanidine, as well as mercapto andseleno derivitives, of which GED is an example. Still further examples of suitable NOS inhibitors include sesquiterpene and guaianolide compounds, such as ergolide and indicanone. Other examples of suitable NOS inhibitors include compounds from medicinal plants such as flavonoids, polyacetylenes, lignans and terpenes.

The pharmaceutical formulations of the present invention need not in themselves contain the entire amount of the agent needed to be effective; as such effective amounts can be reached by administration of a single application or dose, or a plurality of applications or doses of such pharmaceutical formulations.

Pharmaceutical formulations of NOS inhibitors such as those listed above may be suitable for subcutaneous administration such as by injection directly into the affected discs via a needle and/or cannula. In addition to injection, other methods of currently preferred administration include infusion, irrigation and other forms of known parenteral administration. For example, the treatment may be administered to the involved discs by infusion, for example using an infusion pump mechanism. For example, the treatment may be administered to the involved discs by irrigation, for example using arthroscopy.

The formulations may, where appropriate, be conveniently presented in discrete dosage units and may be prepared by any of the methods well known in the art of pharmacy. Such methods may include the steps of bringing into association the active compounds with liquid carriers or finely divided solid carriers or both, and then, if necessary, shaping the product into the desired formulation.

Formulations for parenteral administration include aqueous and non-aqueous sterile injection solutions which may contain anti-oxidants, buffers, bacteriostats and solutes which render the formulation isotonic with the portion of the disc targeted for administration. The formulations may be presented in unit-dose or multi-dose containers, for example sealed ampoules and vials, and may be stored in a freeze-dried (lyophilized) condition requiring only the addition of the sterile liquid carrier, for example, saline water-for-injection, immediately prior to use.

According to one embodiment of the present invention, the therapeutic agents could be delivered by percutaneous intradiscal delivery in a biopolymer substrate. The biopolymer substrate may comprise, for example, hyaluronic acid or a pharmaceutically acceptable salt or ester thereof. The therapeutic could be delivered to multiple spinal segments, and in sequential applications, with the goal of ameliorating proteoglycan loss and concomitant discogenic pain.

The therapeutic agent may be combined with the biopolymer substrate such that the therapeutic agent comprises preferably 0.1 % to 5% of the combined substance, and more preferably 0.1 % to 1.0% of the combined substance.

Alternatively, the therapeutic agents may be administered in conjunction with a surgical procedure, for example, during a surgical procedure such as laminectomy or microdiscectomy. The therapeutic agents may be administer directly to exposed discs

According to one embodiment of the present invention, the therapeutic agents may be administered during a single application. Alternatively, the therapeutic agents may be administered multiple times over a period of between one and 10 days. Treatment may occur once, periodically, or as needed.

Additional references relating to this invention include the following: Inflammatory Mediators as Potential Therapeutic Targets in the Spine, Sally Roberts and Robin C. Butler, Current Drug Targets—Inflammation & Allergy, 2005, 4, 257-266; Possible Pathogenesis of Painful Intervertebral Disc Degeneration, Baogan Peng, et al., SPINE vol.31, no. 5, pp 560-566, 2006; Human Nucleus Pulposis Can Respond to a Pro-inflammatory Stimulus, J. G. Burke, et al., SPINE vol.28, no. 24, pp 2685-2693, 2003; Possible Mechanism of Painful Radiculopathy in Lumbar Disc Herniation, Mamoru Kawakami et al., Clinical Orthopaedics and Related Research, no.351, pp 241-251, 1998; Anti-Apoptotic Effects of Caspase Inhibitors on Rat Intervertebral Disc Cells, Jong-Beom Park, et al., Journal of Bone and Joint Surgery, 2006; Inhibition of Experimental Gingivitis in Beagle Dogs With Topical Mercaptoalkylguanidines, David W. Paquette, et al., J. Periodontol, March 2006; Histochemical Demonstration of Nitric Oxide in Herniated Lumbar Discs, Hiroshi Hashizume, et al., SPINE vol. 22, no. 10, pp 1080-1084, 1997; Cyclic Tensile Stretch Modulates Proteoglycan Production by Intervertebral Disc Annulus Fibrosus Cells Through Production of Nitrite Oxide, Francois Rannou et al., Journal of Cellular Biochemistry 90:148-157, 2003; Nitric Oxide Mediates the Change of Proteoglycan Synthesis in the Human Lumbar Intervertebral Disc in Response to Hydrostatic Pressure, Gen-Zhe Liu, et al., SPINE vol, 26, no. 2, pp 134-141, 2001; Intervertebral Disc Cell Apoptosis by Nitric Oxide: Biological Understanding of Intervertebral Disc Degeneration, Kobe J. Med. Sci. 46, 283/295, December 2000; Herniation of Cervical Intervertebral Disc, Immunohistochemical Examination and Measurement of Nitric Oxide Production, Nobuaki Furusawa et al., SPINE vol. 26, no. 10, pp. 1110-1116, 2001; Nitric Oxide Regulation System in Degenerative Lumbar Disease, Takuya Watanabe et al., Kurume Medical Journal, 52, 39-47, 2005; mRNA Expression on Interleukins Phospholipade A₂, and Nitric Oxide Synthase in Nerve Root and Dorsal Root Ganglion Induced by Autologous Nucleus Pulposus in the Rat, Mamoru Kawakami et al., Journal of Orthopaedic Research, 17:9410946, 1999; The Potential of Gene Therapy for the Treatment of Disc Degeneration, S. Tim Yoon, Orthop Clin N Am 35 pp 95-100, 2004; Gene Therapy Application for Intervertebral Disc Degeneration, Corey J. Wallach et al., SPINE vol. 28, no. 15S, pp S93-S98, 2003; Gene Therapy to Prevent or Treat Disc Degeneration: Is This the Future?, Eric A. Levicoff et al., SpineLine March/April 2005 pp 10-16; Adenovirus-Mediated Gene Transfer to Nucleus Pulposus Cells, Kotaro Nishida, et al., SPINE vol. 23, no. 22, pp 2437-2443, 1998; Emerging Techniques for Treatment of Degenerative Lumbar Disc Disease, Howard An et al., SPINE vol. 28, no. 15S, pp S24-S25; Biologic Treatment for Intervertebral Disc Degeneration, Frank Phillips et al., SPINE vol. 28, no. 15S, p S99, 2003; Biological Repair of Intervertebral Disc, Howard S. An, et al., SPINE vol. 28, no. 15S, pp S86-S92, 2003; Cell Therapy for Disc Degeneration—potentials and pitfalls, Helena Brisby et al. Orthop Clin N Am 35 (2004) 85-93; Intervertebral Disc Cell Therapy for Regeneration: Mesenchymal Stem Cell Implantation in Rat Intervertebral Discs, Gwen Crevensten et al., Annals of Biomedical Engineering vol. 32, no. 3, March 2004 pp. 430-434; Safety Assessment of Intradiscal Gene Transfer: A pilot study, Corey J. Wallach et al., The Spine Journal 6 (2006) 107-112; Sequiterpene Lactones Specifically Inhibit Activation of NF-κB by Preventing the Degredation of IκB-α and IκB-β, Steffen P. Hehner et al., The Journal of Biological Chemistry, vol. 273, no. 3, issue of January 16, pp 1288-1297, 1998; Inhibitory Activity of Chinese Medicinal Plants on Nitric Oxide Synthesis in Lipopolysaccharide-Activated Macrophages, Jae-Ha Ryu et al., The Journal of Applied Pharmacology, 9, pp 183-187 (2001); Cytotoxicity and NMR Spectral Assignments of Ergolide and Bigelovin, Qin Wang et al., Planta Med. 62 (1996); Guaianolides from Viguiera gardneri inhibit the transcription factor NF-κB, Karin Schorr et al., Phytochemistry 60 (2002) pp 733-740; Anti-inflammatory Activity of New Guaiane Type Sesquiterpene from Wikstroemia indicia, Li-Yan Wang et al., Chem. Pharm. Bull. 53(1) pp 137-139 (2005); Ergolide, sesquiterpene lactone from Inula britannica, inhibits inducible nitric oxide synthase and cyclo-oxygenase-2 expression in RAW 264.7 macrophages through the inactivation of NF-κB, Jeung Whan Han et al., British Journal of Pharmacology (2001) 133, pp 503-512; Cytoxic Sesquiterpene Lactones from Inula britannica, Eun Jung Park et al., Planta Med. 64 pp 752-754 (1998); U.S. Pat. No. 5,674,907; U.S. Pat. No. 5,905,089; U.S. Pat. No.5,929,063; U.S. Pat. No. 6,656,925; U.S. Pat. No. 6,747,062; U.S. Pat. No. 6,908,630; and PCT International Patent Application No. WO 03/063799.

The above specific embodiments are illustrative, and many variations can be introduced on these embodiments without departing from the spirit of the disclosure or from the scope of the appended claims. For example, elements and/or features of different illustrative embodiments may be combined with each other and/or substituted for each other within the scope of this disclosure and appended claims.

### EXAMPLES

I. Availability of NOS Inhibitors

Techniques for the synthesis of NOS inhibitors, particularly GED, L-NAME, and L-NMMA are known in the art and such compounds are commercially available, for example, from Alexis Biochemicals, a division of Axxora, LLC of San Diego, Calif.

GED is available from Alexis Biochemicals in the form of GED.bicarbonate [C₆H₁₆N₆S₂.2H₂CO₃]. Product specific literature references include: Spontaneous rearrangement of aminoalkylisothioureas into mercaptoalkylguanidines, a novel class of nitric oxide synthase inhibitors with selectivity towards the inducible isoform, G. J. Southan, et al., Br. J. Pharmacol. 117, 619 (1996); Pharmacological characterization of guanidinoethyldisulphide (GED) a novel inhibitor of nitric oxide synthase with selectivity towards the inducible isoform, C. Szabo, et al., Br. J. Pharmacol. 118, 1659 (1996); and The iNOS inhibitor and peroxynitrite scavenger guanidinoethyldisulfide (GED) prevents onset of type I diabetes in two animal models, G. J. Southan, et al.; Acta Physiol. Scand. 167 Suppl., O-35 (1999).

L-NAME is available from Alexis Biochemicals in the form of L-NAME.hydrochloride [C₇H₁₅N₅O₄.HCl]. Product specific literature references include: L-NG-nitro arginine (L-NOARG) a novel, L-arginine-reversible inhibitor of endothelium-dependent vasodilatation in vitro, P. K. Moore, et al., Br. J. Pharmacol. 99, 408 (1990); and Generation of superoxide by purified brain nitric oxide synthase, S. Pou, et al., J. Biol. Chem. 267, 24173 (1992).

L-NMMA is available from Alexis Biochemicals in the form of L-NMMA.monoacetate [C₇H₁₆N₄O₂.CH₃COOH]. Product specific literature references include: Identification of arginine as a precursor of endothelium-derived relaxing factor, I. Sakuma, et al., PNAS 85, 8664 (1988); and A specific inhibitor of nitric oxide formation from L-arginine attenuates endothelium-dependent relaxation, D. D. Rees, et al., Br. J. Pharmacol. 96, 418 (1989).

II. Efficacy and Dosage Determinaiton

The use of NOS inhibitors, particularly GED, L-NAME, and L-NMMA for the treatment of disc degeneration is tested in porcine subjects to determine efficacy and dosage.

A. Porcine Subcutaneous Pouch Model

For each of the GED-based treatment, L-NAME-based treatment, and L-NMMA-based treatment, a separate test pig is the subject of treatment. Inflammation is induced in the involved discs of a plurality of test pigs. For each treatment, a subcutaneous pouch around each involved disc is created and the compound is injected into the subcutaneous pouch of the respective test pig. Fluid is extracted from the pouch at 6 hours, 24 hours, 48 hours, 7 days, 10 days, and 14 days, and analyzed to determine the concentrations of pro-inflammatory cytokines, nitrites, and ECM components and to determine initial efficacy and to define a dose for Study B below.

B. Inflammation-Induced Disc Degeneration Model

Each of the GED-based treatment, L-NAME-based treatment, and L-NMMA-based treatment is performed in a separate test pig. Inflammation is induced in a pig's disc. For each treatment, following induction of inflammation in intervertebral disc space the compound is introduced. Outcome is observed over 2-12 weeks, and inflammatory cytokines, ECM components, x-rays analyzed to establish efficacy.

The use of NOS inhibitors, particularly GED, L-NAME, and L-NMMA for the treatment of disc degeneration is effectively used to treat human subjects suffering from a disc degeneration disease.

C. Biochemical Injection Treatment in Human Subjects

Patient Selection

Thirty patients with chronic intractable lower back pain and positive discography would participate in the study. These would be adult patients with chronic lower back pain who have been unresponsive or responded poorly to multiple previous methods of treatment, including physical therapy, multiple analgesics, injection therapy, laminectomies, fusions and intradiscal electrothermal annuloplasty (IDET) procedures. These patients would have positive discography at one or more lumbar levels and concordant pain provocation combined with morphologic disc abnormalities.

Composition of Injected Solutions

Using sterile techniques and United States Pharmacopeia-grade pharmaceuticals solutions containing the NOS inhibitors are prepared, specifically, one solution is prepared for each of GED, L-NAME, and L-NMMA. The “GED disc solution” consists of 0.3 mg/kg to 10 mg/kg GED by subject weight; 1.0 to 100 micromolar by concentration. The “L-NAME disc solution” consists of 1.0 mg/kg L-NAME by subject weight; 5.0 to 100 micromolar by concentration. The “L-NMMA disc solution” consists of 1.0 mg/kg L0NMA by subject weight; 5.0 to 100 micromolar by concentration. These concentrations are derived based on solubility and tolerance characteristics of the constituents. These are mixed with 33% nonionic contrast and 33% of 50% dextrose at the time of injection. A total of 1 to 2 cc of each solution is injected into each involved disc. All solutions are tested on separate patients. Injection promptly terminates if there is any leakage of contrast into the epidural space. Zygapophyseal joint injections are performed after fluroscopic confirmation of correct needle placement using intra-articular contrast and are performed at the same levels as the disc injections.

Injection Protocol

To avoid patient discomfort, the first series of injections are performed at the time of diagnostic discography. An intradiscal injection of 1 to 2 cc is used at each involved disc level. The levels are determined by discography. This is combined with injection of the zygapophyseal joints at the painful disc level(s) with the modified solution mixture as described above.

Alternatively, or additionally, the treatment may be administered to the involved discs by infusion, for example using an infusion pump mechanism. Alternatively, or additionally, the treatment may be administered to the involved discs by irrigation, for example using arthroscopy.

Other Treatments

Patients are allowed to continue their ongoing treatment protocols and pain medications as needed.

Assessment of Outcome

The patients subjectively assess their level of pain and disability using a disability questionnaire pertaining to activities of daily living before and after treatment. A score of 0 corresponds to no impact of low back pain on activities of daily living and a score of 24 indicates an extreme level of dysfunction. A standard visual analogue pain scale (0 to 10) is used to determine the patients' subjective estimate of pain before and after treatment. Pre- and post-treatment results are compared and results are obtained. By this approach, each of GED, L-NAME and L-NMMA are effective in treating the patients and providing symptomatic relief to them.

1. A method for treating a vertebrate subject suffering from a degenerative disc disease which comprises administering to the subject an inhibitor of nitric oxide synthase (NOS) in an amount effective to treat the subject.

2. The method of claim 1, wherein the amount effective to treat the subject is an amount effective to slow the progression of the disc degeneration disease.

3. The method of claim 1, wherein the amount effective to treat the subject is an amount effective to prevent the progression of the degenerative disc disease.

4. The method of claim 1, wherein the amount effective to treat the subject is an amount effective to reduce pain concomitant with the degenerative disc disease.

5. The method of claim 1, wherein the inhibitor has the chemical structure: 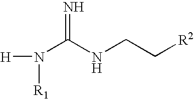
or a salt thereof, wherein
R₁ is C₁-C₆ alkyl, C₂-C₆ alkenyl, C₂-C₆ alkynyl, OH, H, —NO₂, or halide, or a substituted derivative thereof; and
R₂ is independently a substituted or unsubstituted, straight chain C₁-C₃₀ alkyl or branched C₁-C₃₀ alkyl, or a substituted derivative thereof, wherein the alkyl, if substituted, is substituted by hydroxy, carboxy, amino, acetoxy, or nitroxy.

6. The method of claim 1, wherein the inhibitor has the chemical structure: 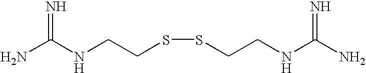
or a pharmaceutically acceptable salt or ester thereof.

7. The method of claim 1, wherein the inhibitor has the chemical structure: 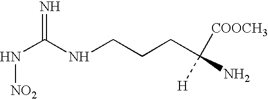
or a pharmaceutically acceptable salt or ester thereof.

8. The method of claim 1, wherein the inhibitor has the chemical structure: 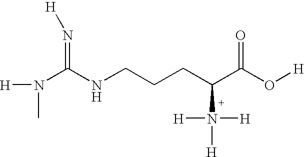
or a pharmaceutically acceptable salt or ester thereof.

9. The method of claim 1, wherein the inhibitor is administered as a component of a composition which comprises a pharmaceutically acceptable carrier.

10. The method of claim 9, wherein the pharmaceutically acceptable carrier comprises a biopolymer.

11. The method of claim 10, wherein the biopolymer comprises hyaluronic acid or a pharmaceutically acceptable salt or ester thereof.

12. The method of claim 9, wherein the inhibitor is present within the carrier at a level of 0.1% to 5%.

13. The method of claim 9, wherein the inhibitor is present within the carrier at a level of 0.1% to 1.0%.

14. The method of claim 1, wherein the inhibitor is administered in conjunction with a surgical procedure which results in the intervertebral discs of the subject being accessible to the inhibitor.

15. The method of claim 14, wherein the surgical procedure comprises laminectomy or microdiscectomy.

16. The method of claim 1, wherein the inhibitor is administered by means of a needle or a cannula.

17. The method of claim 6, wherein the amount of the inhibitor administered is between 0.1 mg/kg and 30 mg/kg body weight of the subject.

18. The method of claim 6, wherein the amount of the inhibitor is administered between 0.3 mg/kg and 10 mg/kg body weight of the subject.

19. The method of claim 6, wherein the amount of the inhibitor administered is between 0.1 to 1000 micro molar by concentration.

20. The method of claim 6, wherein the amount of the inhibitor administered is between 1.0 to 100 micro molar by concentration.

21. The method of claim 7, wherein the amount of the inhibitor administered is between 1.0 mg/kg and 30 mg/kg body weight of the subject.

22. The method of claim 7, wherein the amount of the inhibitor is administered between 1.0 mg/kg and 10 mg/kg body weight of the subject.

23. The method of claim 7, wherein the amount of the inhibitor administered is between 1.0 to 1000 micro molar by concentration.

24. The method of claim 7, wherein the amount of the inhibitor administered is between 5.0 to 100 micro molar by concentration.

25. The method of claim 8, wherein the amount of the inhibitor administered is between 1.0 mg/kg and 30 mg/kg body weight of the subject.

26. The method of claim 8, wherein the amount of the inhibitor is administered between 1.0 mg/kg and 10 mg/kg body weight of the subject.

27. The method of claim 8, wherein the amount of the inhibitor administered is between 1.0 to 1000 micro molar by concentration.

28. The method of claim 8, wherein the amount of the inhibitor administered is between 5.0 to 100 micro molar by concentration.

29. The method of claim 1, wherein the inhibitor is administered multiple times over a period of 30-180 days.
